(19) United States
(12) Patent Application Publication
(10) Pub. No.: US 2005/0112695 A1
(43) Pub. Date: May 26, 2005

(54) Method of detecting bovine spongiform encephalopathy

(76) Inventor: Donald W. Meyer, Ixonia, WI (US)

Correspondence Address: REINHART BOERNER VAN DEUREN S.C.;ATTN: LINDA GABRIEL, DOCKET COORDINATOR, 1000 NORTH WATER STREET, SUITE 2100, MILWAUKEE, WI, 53202, US

(21) Appl. No.: 10/982,753

(22) Filed: Oct. 27, 2004

Related U.S. Application Data
(60) Provisional application No. 60/515,803, filed on Oct. 27, 2003.

(51) Int. Cl. G01N 33/53; G06F 19/00; G01N 33/48; G01N 33/50; G01N 33/00

(52) U.S. Cl. 435/7.1; 436/86; 702/19

(57) ABSTRACT

A method for diagnosing bovine spongiform encephalopathy (BSE) in live or postmortem bovine animals prior to the onset of clinical BSE symptoms including detecting spectral changes between BSE-positive samples and BSE-negative samples thereby providing a predictive model and diagnostic test for classifying unknown samples as disease-positive or disease-negative.

### CROSS REFERENCE TO RELATED APPLICATIONS

This application claims priority of provisional application Ser. No. 60/515,803, which is entitled “Method of Detecting Transmissible Spongiform Encephalopathies”, and which was filed on Oct. 27, 2003, the entirety of which is hereby incorporated herein by reference.

### BACKGROUND OF THE INVENTION

Field of the Invention—The present invention relates to methods for detecting a change in spectral response between normal and diseased tissue samples as an indication of a bovine spongiform encephalopathy infection, and more particularly, to BSE diagnostic methods including comparing spectral data obtained from an “unknown” sample to a calibration model comprising spectral data obtained from BSE-normal and BSE-diseased samples.

Mad cow disease, or bovine spongiform encephalopathy, is a chronic degenerative disease that affects the central nervous system of cattle. Bovine spongiform encephalopathy was discovered in the mid-1980's in the United Kingdom, and belongs to a family of diseases known as transmissible spongiform encephalopathies.

Transmissible spongiform encephalopathies (TSEs), also referred to as prion diseases, are a family of contagious, degenerative neurological diseases found in humans and animals. A TSE infects the central nervous system of a host species and eventually attacks the brain of the host species. TSEs have a long incubation period, and once infected, a host species may not exhibit clinical symptoms for years. Clinical symptoms of TSEs can include emaciation, loss of bodily condition, loss of coordination and movement, excessive salivation, and/or loss of bodily functions. While the clinical symptoms of each specific transmissible spongiform encephalopathy (TSE) may vary somewhat from species to species, TSEs always cause death in infected victims.

TSEs are characterized by tiny holes or lesions in the brain that give the brain a sponge-like appearance and are associated with the aggregation in the brain of prions, an abnormal form of a naturally occurring, membrane protein called a prion protein (PrP). Normal prion protein molecules, designated as PrP or PrP^{c} (cellular), are predominantly found on the surface of neuronal cells and in spinal fluid as well as on the surface of certain other non-neuronal cell types such as lymphoreticular tissue. The normal PrP^{c} is sensitive to digestion with specific enzymes.

Disease-associated prions are defined as small, proteinaceous infectious particles that resist inactivation by procedures that modify nucleic acids. These infectious prions, designated PrP^{res} (protease resistant protein), are characterized as abnormal, insoluble isoforms of normal mammalian PrP^{c}.

Normal PrP^{c} and infectious PrP^{res} share the same amino acid sequence and have almost identical molecular weights of 33 to 35 kd (6033858), but differ from each other in their secondary structure. Normal prion proteins contain a predominantly alpha helix structure and contain almost no beta sheet structure. It is believed that the difference between normal PrP^{c} and infectious PrP^{res} is conformational, with infectious PrP^{res} containing a significantly higher beta-sheet content than normal PrP^{c}. The normal prion protein's usual function is unknown, but its shape can be converted into the abnormal, beta-sheet rich disease-associated form. Once the abnormal prions are present, it is suggested that the abnormal prion protein spreads in a chain reaction by converting normal prion molecules into the abnormal form, i.e. converting PrP^{c} to PrP^{res}.

The pre-clinical phase of a TSE is the long incubation period after infection with PrP^{res} in which the species exhibits no external clinical symptoms of the disease. However, it is known that the presence of PrP^{res}, or a precursor thereof, can be detected in the lymphoreticular system (tonsils, lymph nodes, eyes, spleen, etc.) and in other tissues/biological materials such as the brain, muscles, blood and spinal cord fluid in species susceptible to TSEs. For example, because infectious prions, PrP^{res}, are protease resistant, it is suggested that prions accumulate in the lymph system or nervous system of an animal species, in addition to the brains of infected the host, where they aggregate into distinctive filaments or plaques on the surface of the cell membrane.

Prions are transmitted from one host to another; however, they do not exhibit many other features of traditional infective agents, such as bacteria, viruses, or fungi. As such, prions are referred to as infectious “agents” of disease. How a prion moves from species to species is somewhat unclear; however, it is known that a TSE can be transmitted from animal to animal contact, and that animals exposed to a TSE contaminated environment, infected tissue, body fluids, infected carcasses, or contaminated medical instruments may also become infected. Once the infectious agent enters the brain of the host, it can lie dormant for several years, but when it is activated, the abnormal prion molecules kill brain cells leaving large areas of spongy holes in the brain, and leaving large clumps of abnormal prion protein in the brain. Once the abnormal prion is activated, the disease can run its course in less than a year, causing death. Further, it has also been discovered that routine sterilization procedures, such as boiling or irradiation, do not prevent transmission of TSEs. Further, cleaning chemicals that one would use to disinfect surfaces for bacteria or viruses such as Lysol, or Betadine, are not effective in destroying the infectious capability of the prions.

It is likely that most mammalian species can develop TSEs, and several specific prion diseases are identified. For example, scrapie, a disease that has been well known for several centuries, is the infective TSE found in goats or sheep. Chronic wasting disease (CWD) is a TSE found in free ranging and captive mule deer, white tailed deer and elk across North America. Human infective TSEs include Creutzfeld-Jakob disease (CJD), kuru, fatal familial insomnia (FFI), and Gerstmann-Straussler-Scheinker disease (GSS). In addition, human infective TSE called new variant Creutzfeld-Jakob disease (nvCJD) was identified in 1996, and has been found in the United Kingdom and several other European countries. Other TSEs also include feline spongiform encephalopathy (FSE) in cats and transmissible mink encephalopathy (ILE) in mink.

Moreover, it has been suggested that food products containing TSE infected tissue may cause cross-species forms of a transmissible spongiform encephalopathy disease. For example, it is believed that in the 1980's, feed contaminated with scrapie-infected sheep tissue and/or sheep by-products was fed to cattle causing BSE infections.

Moreover, in the 1990's, the British government concluded that new variant Creutzfeld-Jakob (vCJD) disease in humans was caused by ingesting meat from BSE infected cows. Indeed, as of December 2003, one hundred and fifty-three people have contracted vCJD through the ingestion of infected meat or bovine products. There is presently no known cure for this disease, thus, efforts at disease prevention have primarily focused on identification and destruction of infected animals, restriction on exports of products from suspected infected animals and overall restrictions on export of such animals. These restrictions have resulted in significant economic loss to producers, ranchers and cattle renderers.

Most recently, BSE infections have been discovered in the United States and Canada. BSE has a lengthy, silent incubation period compared to the life span of the animal, which range from 30 months to eight years. However, diagnosis of BSE during the incubation period has not been possible in traditional BSE tests. Indeed, postmortem analysis of bovine brain tissue, after the incubation period—when the animal is already exhibiting clinical symptoms of the disease is not effective in controlling the spread of vCJD or BSE. Thus, with a U.S. cattle population of 45 million, early detection of BSE is critical to the livestock, dairy, and bovine products industries.

Current Methods of Detection

Diagnosis of bovine transmissible spongiform encephalopathy involves several different methods.

Postmortum microscopic or histological examination of brain tissue, especially the medulla oblongata at the obex, is still routinely performed as a method of confirming a BSE case. However, such method requires the expertise of laboratory technicians and is time consuming, and therefore, not a practical method of testing a large volume of tissue samples. In addition, microscopic examination of brain tissue detects lesions in the brain occurring only after the onset of the clinical disease, but is not used to detect early stages of infection with the disease.

The Western Blot assay, and test variations thereof, for the detection of a BSE relies on the differential sensitivity of the infectious prions, PrP^{res}, to protease K and thus, can be a quantitative measure of the presence of infectious prions. Tissue samples, for example, brain or lymph tissue, are first digested with protease K or an antibody that can discriminate between normal cellular PrP^{c} of the specific host species and the protease resistant form PrP^{res}. The sample is denatured and diluted to an end point where PrP^{res} can no longer be detected in SDS PAGE gel. While Western Blot is considered a reliable, gold standard test for the detection of prion proteins, and therefore, a diagnosis for BSE, the Western Blot test is considered technically demanding, involves careful and inefficient preparation of samples, and required the use of expensive and environmentally sensitive monoclonal antibodies. Accordingly, improper analytical techniques, instrumentation malfunctions, biased calculations, or inconsistent sample preparation techniques from technician to technician can result in resulting in errors in the test, and therefore, result in the possibility in error in test results. Because of these difficulties and because Western Blot testing cannot be automated, it is not an appropriate test for the rapid, routine diagnosis, confirmation or screening of samples for BSE.

BSE testing is also performed by immunohistochemistry assay of formalin fixed tissues. Although IHC is a sensitive and specific diagnostic test, the assay requires careful collection and trimming of tissues, formalin fixation, processing and sectioning, IHC assay, and interpretation of results by a pathologist. The turnaround time is minimally measured in days and laboratory capacity is limited. In addition, tissue preparation is lengthy and subject to operator error. As with the Western Blot assay, use of expensive PrP^{res} specific monoclonal antibodies is a further disadvantage to implementing this type of testing in a large-scale application. Further, the nature of IHC testing makes diagnosis of BSE at the early stages of the disease subject to operator error or inconsistency from operator to operator.

Recently, the U.S.D.A. and several European nations have adopted the use of a rapid, enzyme-linked immunosorbent assay, or ELISA, test to detect the presence of the protease-resistant PrP^{res}. This test utilizes brain tissue which must be harvested and used within 30 days. Sample preparation can take up to one hour involving alteration and treatment of the sample with several buffer solutions and use of environmentally sensitive monoclonal antibodies. These tests produce results in 4 to 5 hours and cost up to twenty dollars per test kit.

Currently, there are no approved live-animal tests for BSE. In addition, there are no available tests for testing meat or other muscle tissue for BSE.

IR spectroscopy can be used to measure the absorbance/reflectance of infrared light in a sample. Specific absorbance peaks can be correlated to the presence of specific functional groups, chemical bonds and/or structural features of a compound in the sample, including water, proteins, or lipids. The absorbance patterns of samples can be compared to a reference (calibration) set to identify peaks specifically associated with a group, bond or structural feature of interest. IR analysis, alone or in conjunction with a fourier transform function (FTIR), is a standard tool in some prion research laboratories, in which the difference in protein conformation between the normal cellular prion protein and the disease-associated prion were detected. Diagnostic use of the method has been investigated in a hamster model of scrapie. However, application of the technique to naturally occurring bovine spongiform encephalopathy has not been described. Moreover, these applications utilize FTIR microscopy in conjunction with an FTIR spectroscopy instrument, requiring time consuming sample preparation, long scanning times and significant operator training. Further, in conjunction with the prior, the cost of FTIR microscopy equipment and required accessories discourages routine, widespread use of these methods in the diagnosis of BSE.

### BRIEF DESCRIPTION OF THE DRAWINGS

FIG. 1A illustrates a lymph node, identifying the cortex and paracortex regions and the primary and secondary follicles contained therein;

FIG. 1B illustrates a brain and brain stem, identifying the obex portions utilized in the present invention;

FIG. 1B illustrates the brain stem, obex and spinal cord tissues utilized in the methods of the present invention;

FIG. 2A is a flowchart depicting the development of a calibration model according to the methods of the present invention;

FIG. 2B is a flowchart depicting the diagnostic test method for diagnosing BSE in an unknown sample according to the methods of the present invention;

FIG. 3 is a scatter plot of the Mahalanobis distances for each calibration and validation sample in Example 1 demonstrating a “BSE-positive” cluster or “BSE-negative” cluster for bovine blood serum samples;

FIG. 4 is a results table presenting the calibration and validation results of Example 1 and plotted in FIG. 3;

FIG. 5 is a scatter plot of the Mahalanobis distances for each calibration and validation sample in Example 2 demonstrating a “BSE-positive” cluster or “BSE-negative” cluster for bovine lymph tissue samples;

FIG. 6 is a results table presenting the calibration and validation results of Example 2 and plotted in FIG. 5;

FIG. 7 is a scatter plot of the Mahalanobis distances for each calibration and validation sample in Example 3 demonstrating a “BSE-positive” cluster or “BSE-negative” cluster for bovine brain tissue samples;

FIG. 8 is a results table presenting the calibration and validation results of Example 3 and plotted in FIG. 7; and

FIG. 9 is a results table presenting the calculated score for each of the samples in Example 3.

### SUMMARY OF THE INVENTION

In light of the forgoing, there is a need for a rapid, inexpensive and reliable method for detecting a bovine spongiform encephalopathy (BSE) in a high throughput environment in order to increase the efficiency of surveillance and containment efforts.

Accordingly, it is an object of the present invention to use an IR spectroscopic method, such as Fourier transform infrared spectroscopy (FTIR), as a rapid test for detecting naturally occurring bovine spongiform encephalopathy (BSE) in live and/or postmortem bovine species, thereby overcoming various deficiencies and shortcomings of the prior art, including those outlined above. It is a further object of the invention to provide a calibrated spectrometric system for detecting spectral changes between BSE-positive (diseased) and BSE-negative (normal) samples in unadulterated, untreated tissue and/or biological fluid samples, thereby providing a means for diagnosing BSE. It can be another object of the present invention to provide a method of confirming a previously obtained BSE diagnosis determined through use of a conventional technique such as a Western Blot or ELISA assay.

Thus, it is a related object of the present invention to provide a method for diagnosing mad cow disease in any live or postmortem bovine species prior to the onset of the clinical symptoms of a BSE infection, regardless of BSE strain or animal genotype. Such methods utilize IR spectroscopy to detect spectral changes between naturally occurring BSE-positive and BSE-negative brain tissue, lymphoreticular system tissue and/or blood samples, providing a calibrated system and method for detecting a BSE infection in unknown brain tissue, lymphoreticular system tissue and/or blood samples.

It is another object of the present invention to provide a rapid, primary analytical method for diagnosing a BSE infection that utilizes unadulterated, untreated (e.g., fresh or previously frozen) samples, eliminating the need for technically difficult and time consuming mechanical and/or chemical preparation of the sample before a diagnostic result can be obtained. Accordingly, it can be a further object of the present invention to provide a primary test for the diagnosis of a BSE infection, wherein a misdiagnosis of BSE is minimized by eliminating systematic and random errors that occur in sample preparation, testing, and interpreting the results of currently known methods. Ergo, it can be an object of the present invention to provide a more reliable and predictable method for diagnosing a BSE infection.

It is an object of the present invention to reduce the time required to not only prepare the sample, but perform analysis of the sample so that high-volume, rapid throughput testing of samples can be performed easily by governments and researchers. Thus, because there is no subjectivity in the analysis of the spectral information, operator fatigue will not affect the outcome of the analysis. It is therefore a further object of the present invention to minimize the costs of analyzing samples for a BSE wherein there are no expensive chemicals or antibodies needed in sample preparation, the operators require no special training, and the instrumentation is inexpensive compared to traditional methods of detection.

It can be a further object of the present invention to provide a rapid, fully automated method for classifying an animal as BSE-positive or BSE negative, wherein each of a plurality of samples is retained in a receptacle and tagged corresponding to the age, type and location of the animal, and wherein each sample is automatically transferred to the FTIR spectroscopic instrument, scanned, analyzed with respect to a calibration model and classified as BSE-positive or BSE negative. The method further includes sanitization of the FTIR spectroscopic instrument in between testing of individual samples. In addition, the method can include automatic generation of results reports for each sample and/or groups of samples.

It will be understood by those skilled in the art that one or more aspects of this invention can meet certain objectives, while one or more other aspects can meet certain other objectives. Each objective may not apply equally, in all instances, to every aspect of the present invention. As such, these objectives can be viewed in the alternative with respect to any one aspect of the present invention.

Other objects, features, benefits and advantages of the present invention will be apparent from the foregoing, in light of the summary and the examples and descriptions which follow, and will be readily apparent to those skilled in the art having knowledge of various prion detection systems and BSE diagnosis methods, conventional sample preparation techniques, and subsequent application. Such objects, features, benefits and advantages will be apparent from the above as taken in conjunction with the accompanying examples, tables, graphs, data and all reasonable inferences to be drawn therefrom.

Without limitation to any particular theory or mode of operation, the spectrometric method described herein identifies one or more constituent functional groups and/or structural features of a molecular compound or composition evidenced by one or more spectra from a diseased sample corresponding to either the presence of the diseased prion protein, PrP^{res}, in the diseased tissue sample, or another chemical, molecular and/or structural alteration occurring within the sample as a precursor to the onset of clinical BSE symptoms. Thus, spectral differences between normal and diseased tissue and/or blood samples can be observed before BSE-induced lesions are observed in the brain of the bovine animal. By developing a calibration model, these spectral distinctions provide a method for differentially diagnosing naturally occurring BSE in unknown samples. The spectrometric method used may be absorption, reflectance, emission or transmission spectrometry. Preferably, absorption or reflectance spectra are obtained from a sample using Fourier transform infrared spectroscopy (FTIR) in order to provide a test wherein there is substantially constant pathlength through the sample, and therefore, more consistency from sample to sample and therefore, in the test results.

Sample Preparation

Certain embodiments of the present invention utilize blood samples obtained from a live bovine subject. (Reference is made to Example 1). Blood samples include not only whole blood, but also blood components such as plasma and blood serum. Blood components such as plasma or serum may be separated by conventional methods as are well known to those skilled in the art.

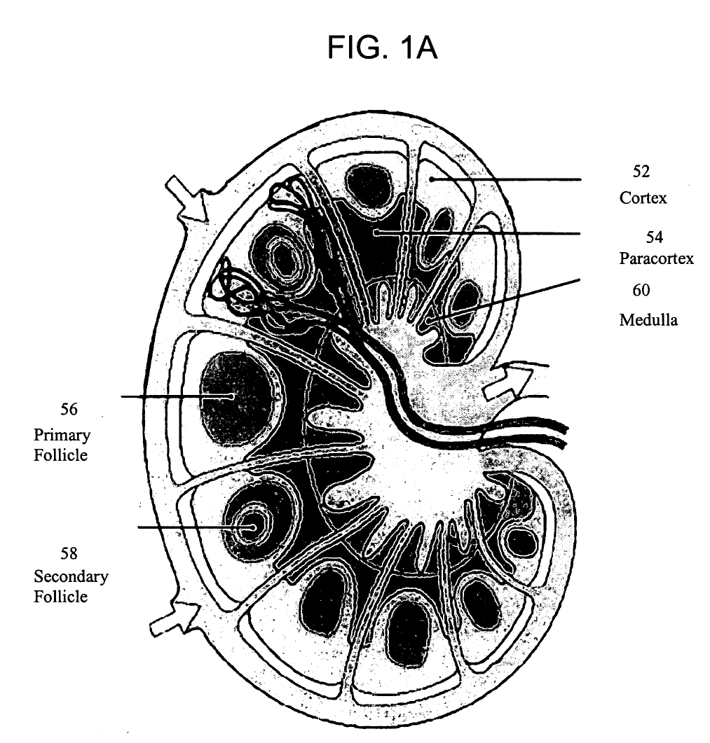

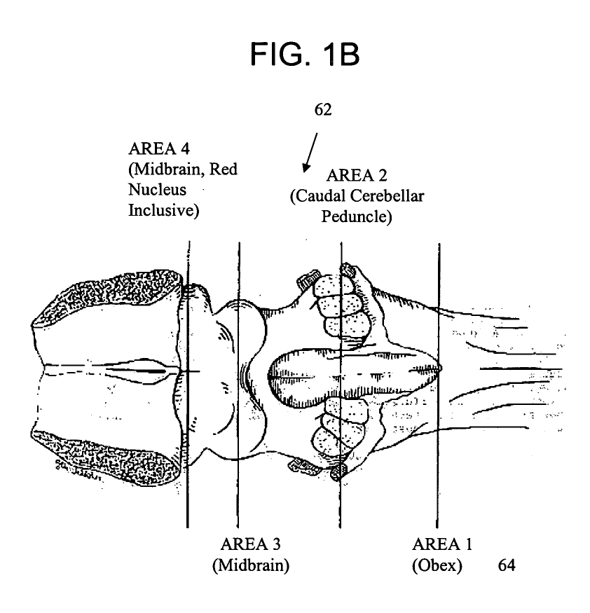

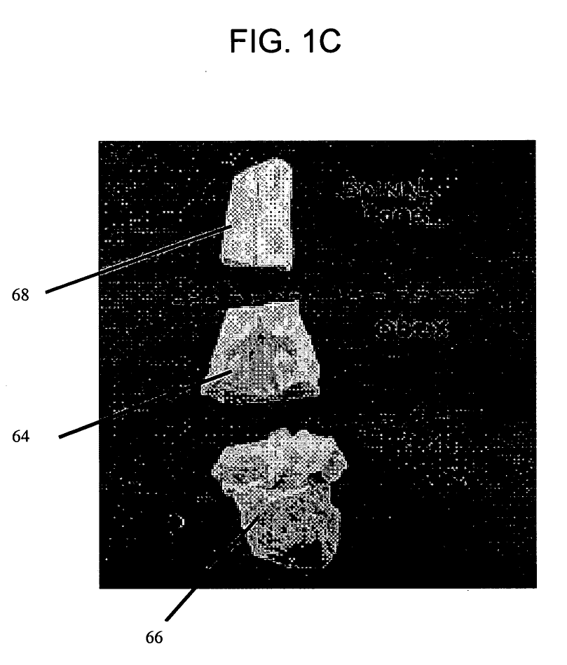

In another embodiment of the present invention, and with reference to Example 2, test samples are obtained from the lymphatic system of a bovine animal. As illustrated in FIG. 1A, lymph tissue samples are obtained by sectioning a lymph node 50 to expose the cortex 52 or paracortex 54 regions of the node in order to preferably obtain a sample section that includes primarily primary 56 and/or secondary follicles 58, but which minimizes medullary tissue 60 within the sample. Preferably, mesenteric lymph node tissue is obtained. It will be apparent to those skilled in the art that any lymphatic system tissue may be used in the present invention, including but not limited to, any lymph node, spleen, lymph nodules (tonsils), thymus, appendix, Peyer's patches and/or small intestines.

In yet another embodiment of the present invention, and with reference to Example 3, central nervous system tissues may be utilized. In particular, as illustrated in FIGS. 1B and 1C, brain tissue 62, such as obex tissue 64 of the bovine animal may be obtained for use in the present invention. In addition, brain stem 66, spinal cord tissue 68 and/or spinal fluid may also be used.

However, consistent with the broader aspects of the invention, suitable tissue samples include additional organ tissues, including but not limited to eye tissue, pancreatic tissue, muscle tissue, tongue nerve cells and/or bone marrow. In particular, any bovine tissue demonstrated to contain PrP^{res} or known to those skilled in the art to contain a BSE-indicating molecular alteration, (for example, muscle tissue, eye tissue, etc.) may be utilized in the methods of the present invention. In addition, other biological fluids known to those skilled in the art to contain PrPᵣₑₛ or another TSE-indicating molecular alteration may be utilized. As such, suitable samples can include tears, milk, saliva, semen, or sweat. Further, suitable samples may also include bodily waste such as urine, feces, or menstrual blood. As described herein, a “sample” refers to a tissue sample, blood and/or a biological fluid sample as previously described.

The present invention includes samples obtained from live or post-mortem bovine animals acquired from farms, slaughtering facilities, livestock auctions, veterinary laboratories, animal feed facilities and/or rendering plants.

As described herein “untreated” samples include samples involving substantially no work-up (chemical, physical or mechanical manipulation) prior to testing. Untreated samples can include fresh and/or thawed samples and also include raw and/or unadulterated samples provided for testing involving substantially no sample preparation other than obtaining the proper amount of the sample. In particular, as distinguishable from the prior art, untreated samples are not homogenized or mixed with water, subjected to enzyme action or chemically or physically extracted.

Preferably, samples are utilized untreated and are neither frozen, heated, nor dried prior to testing. However, samples may be frozen at conventional storage temperatures, e.g. −35° C. or −70° C., or as required by the particular laboratory or testing facility. Thawed samples are cut and/or thinly sectioned to expose a new surface to the spectrometric test instrumentation prior to testing.

In preparation for testing, thin sample sections of approximately about 1 cm×about 1 cm×about 0.2 cm in dimension are obtained from the bovine animal. Sample sections of up to about 3 cm×about 3 cm×about 0.5 cm may also be obtained, depending on the type of sample used, particular requirements of the spectrometric instrumentation and testing accessories employed in performing the methods of the present invention. Where blood or other biological fluids are used, samples from approximately about 10 μl to about 1 ml are obtained from the subject. As will be readily apparent to those skilled in the art, sample size is dependent on not only specific test instrumentation but also on the availability of the sample and the need for further testing of the sample.

Any of the aforementioned sample types may be used to develop a calibration model for a bovine animal and can include different breeds/varieties of such bovine animals. For example, a single bovine calibration/predictive model can include samples from cattle varieties such as, but not limited to, Holstein, Piedmont, and Guernsey varieties in order to classify any type of bovine as BSE-positive or BSE-negative. In addition, a single calibration model can be developed for a specific breed/variety of bovine (e.g. a Holstein calibration model/diagnostic test, a dairy cattle calibration model, a beef cattle calibration model/diagnostic test, a gender-specific calibration model). In addition, a calibration model may be developed for bovine animals in a particular country, region, gender and/or in a particular age-group, depending on specific testing parameters. Thus, it will be apparent to those skilled in the art that the present methodology is unlimited as to either bovine animal genotype or strain of BSE.

The type of unknown sample tested using the methodology of the present invention (e.g. blood, lymph tissue, or brain tissue) is preferably the same type of sample used for developing the calibration model. In addition, it is preferred that each sample used in a particular calibration model or used for a particular diagnostic test be stored at similar temperatures. For example, calibration data sets developed with samples stored at −70° C. temperatures prior to testing should be used to test unknown samples stored at −70° C. However, samples stored at, for instance, −35° C. and fresh samples may be used to develop the same calibration model—which can be used to test unknown samples stored at −35° C. and fresh samples.

The raw, untreated samples are placed directly on the optical bench and a complete scan of the sample is rapidly obtained. Because sample preparation is minimal, samples tested by the present method can be subsequently analyzed and/or confirmed by immunohistochemistry (IHC), Western Blot, ELISA or another validated gold standard test for detecting BSE.

Development of a Calibration Model.

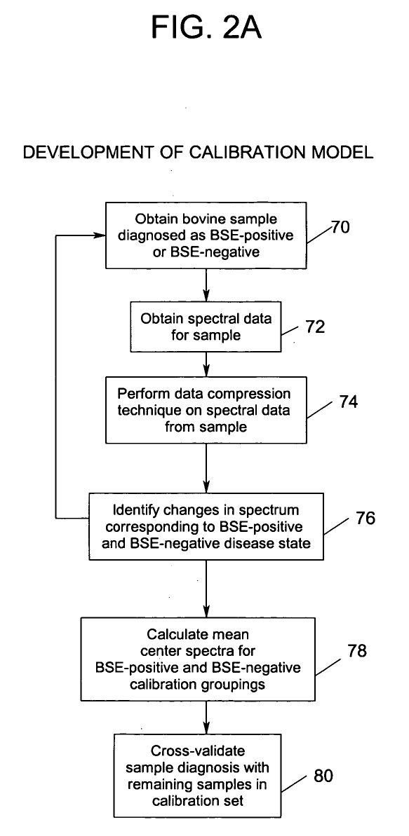

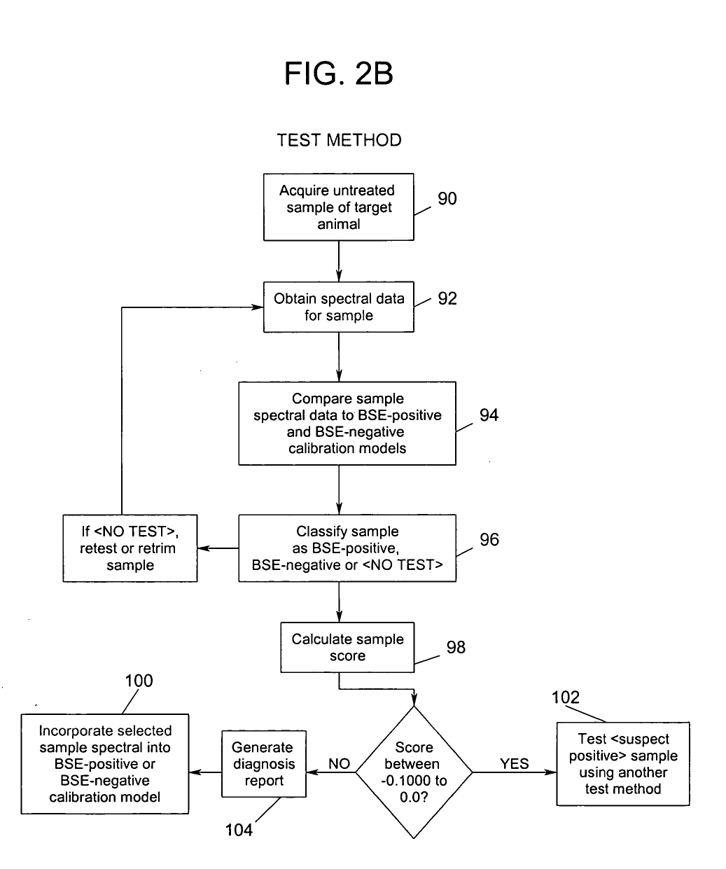

As illustrated in FIG. 2A, to develop a calibration model, a calibration set of infrared absorption data (or alternatively reflectance data) is obtained from samples extracted from bovine subjects that have previously been identified as BSE-positive or BSE-negative, step 70. The infrared absorbance spectra for each of the known samples are obtained, step 72, at wavenumbers from about 7400 cm⁻¹ to about 350 cm⁻¹, preferably from about 4500 cm⁻¹ to about 350 cm⁻¹, and more preferably from about 4000 cm⁻¹ to about 500 cm⁻¹. Without limitation, a more discreet wavenumber range from about 1700 cm⁻¹ to about 1600 cm⁻¹ may also be used to develop a calibration model—wherein such a wavenumber region includes at least a portion of the spectral changes observed between BSE-positive and BSE-negative samples. Thus, consistent with the broader aspects of the present invention, absorbance spectra may be obtained at any range of wavenumbers or wavelengths, or may be obtained in a more discrete range of wavenumbers or wavelengths, depending on the type of sample, the spectrometric method used, and/or the particular bovine animal and/or herd population be diagnosed.

The calibration/reference set of infrared absorption data can be prepared from any number of reference samples, however, the calibration/reference data set will preferably include greater than about 10 reference samples.

Each sample in the calibration set can be scanned multiple times and co-added to increase the signal to noise ratio, thereby enhancing the spectral absorbance features of the sample (that is, the spectral features correlated in the frequency/wavelength domain). For example, tissue samples may be scanned approximately about 64 times, while biological fluids may be scanned approximately about 16 to about 32 times. One skilled in the art will appreciate that a sample may be scanned as many times as necessary to sufficiently distinguish/enhance the correlated spectral features from spectral noise (which shows no correlation in the frequency/wavelength domain). Generally, the more spectra included in the calibration set, the better chance there is of accounting for sample variations/impurities, instrument drifts and changes, and/or operator mistakes; thus, the test will be more sensitive. Irrespective of the number of multiple scans collected for a given sample, it is contemplated by the present invention that complete scanning of a single sample (including multiple scans of the same sample) can be performed, preferably, in less than about one minute.

After each calibration/reference sample is scanned and the respective spectra collected, corresponding calibration models characteristic of a BSE-positive sample and a BSE-negative sample are developed from the calibration spectral data.

Preferably, prior to performing any of the data analysis methods described herein, original spectra is converted to second derivatives to enhance spectral details, such as shoulders of IR bands and to minimize or eliminate baseline shifts. However, the original spectra and/or first derivatives can be used in the spectral analysis for development of the predictive model, as will be well known to those skilled in the art. Data smoothing may be performed using a range of Savitzky-Golay algorithms or by weighted averaging the data using a set of weights that are a normalized Gaussian distribution. Further, any other data smoothing technique known to those skilled in the art may be used.

It will also be readily apparent to those skilled in the art that the data may first be mean centered, linearized, or otherwise normalized before such data analysis. Normalization of spectra may also be required when the calibration data/diagnostic test is transferred to a different spectrometric instrument (i.e. a different testing location), or where samples are tested on different spectrometric instruments. For example, Multiplicative Scatter Correction to account for pathlength variation can be performed prior to utilizing one of the data analysis methods referenced herein.

In order to reduce the amount of spectral data for analysis, the original spectra obtained for each of the known BSE-positive calibration samples are averaged and the original spectral obtained for each of the known BSE-negative samples are averaged. The average spectra for the BSE-positive samples and the average spectra for the BSE-negative samples are subtracted and/or the average spectra are superimposed in a manner that highlights the wavenumber regions exhibiting the most prominent spectral distinctions between the average BSE-positive and the average BSE-negative spectra. These spectral regions are then selected for further analysis/discrimination between the spectra of BSE-positive and BSE-negative samples. Consistent with the broader aspects of the present invention, data reduction can be performed on original (raw) spectra, first or second derivative spectra, data smoothed and/or normalized spectra.

In addition, it will be apparent to those skilled in the art that the entire spectral range of wavenumbers obtained during scanning of the calibration samples can be used in the methodology of the present invention, without utilizing a data reduction technique, provided the data analysis method utilized is capable of sufficiently discriminating between BSE-positive and BSE-negative samples in the calibration set such that unknown BSE-positive and BSE-negative samples can be accurately diagnosed.

Preferably, the method utilizes principal component analysis (PCA) as a chemometric method for breaking down spectroscopic data into its most basic variations, step 74. This type of multivariate technique/spectral data compression technique analyzes entire regions of a spectrum and allows discrimination between the spectra of different groups of samples. In particular, PCA data transformation converts a set of correlated spectral features/variables (the raw spectral data) into a compressed smaller set of uncorrelated variables. This transformation rotates the coordinate system, resulting in the alignment of information on a fewer number of axes than in the original spectral arrangement. This results in a compression of the variables by allowing those variables that are highly correlated with one another to be treated as a single entity. After using PCA, there will be a small set of uncorrelated variables representing most of the information that was in the original set of variables; however, the information will be easier to use in subsequent analytical models.

Consistent with the broader aspects of the invention, any mathematical data analysis technique, spectral data compression technique, predictive modeling method, and/or chemometric analysis method known to those skilled in the art can be used to identify spectral correlation corresponding to a BSE-positive and a BSE-negative disease state within the calibration data set, provided the data analysis technique is capable of sufficiently discriminating between BSE-positive and BSE-negative samples in the calibration set such that unknown samples can be accurately diagnosed. Such techniques can include but are not limited to, Partial Least Squares Regression (PLS), Principal Component Regression (PCR), Multiple Linear Regression (MLR) and Discriminant Analysis. It will at once be appreciated by those skilled in the art that any of the data smoothing, normalization, statistical methods and/or data analysis techniques described herein can be performed using a commercially-available computer software program. Such programs can include, but are not limited to OMNIC® (2002 Thermo Electron Corporation) including TQ Analyst® software (from Thermo Electron Corporation, Madison, Wis., U.S.A.); or PLSpluSIQ™ and GRAMS/AF™ (from Thermo Galactic, Salem, N.H., U.S.A).

Accordingly, principal component analysis identifies patterns in the calibration/reference set spectra that contribute the most to the variation among that group, resulting in mathematical spectra (called loading vectors or principal components) which represent the most common variations to all the calibration data, step 76. The first principal component is constructed to account for as much variability as possible; this often corresponds to the major visual differences among spectra as caused by light scatter, path length changes, or particle size differences. The second principal component is constructed to account for as much of the remaining variability as possible. This often corresponds to the largest absorbance band changes, such as moisture. Additional components will include variation in other bands. A discussion of the underlying theory and calculations of principal component analysis can be found, for example, in Fredericks, et al., Applied Spectroscopy, 39, 303 (1985); Aries, et al., Spectroscopy, 5, 41 (1990); and Haaland, et al., Analytical Chemistry, 60, 1193 (1988).

The amount of variability accounted for in each principal component depends on the specific TSE, the type of sample, and the number of samples in the calibration set. Each time a calibration set is developed, optimized or expanded, the number of principal components used for the predictive model can fluctuate, as needed, to account for variability approaching about 100%. Thus, in order to develop a sensitive calibration model, the number principal components used may account for as much variability in the calibration spectra as possible and/or as practical. Typically, 2 to about 25 principal components will account for about 85% to greater than about 99.9% of the variance of the variables. However, up to about 50 or more principal components can be used, depending on the type of sample used and the variability thereof.

The principal components are then used to predict the disease-state of “unknown” samples. To do this, a set of scaling coefficients called principal component scores (“PC scores”), for each principal component, are calculated for each sample in the calibration set. When the PC scores are multiplied by the principal components, and the results are summed, the original spectra are reconstructed. By knowing the set of principal components, the PC scores will represent the spectra as accurately as the original spectral responses at all the wavelengths considered. Thus, scatter plots of the PC scores over the principal components can provide a means for visualizing and summarizing the spectral data.

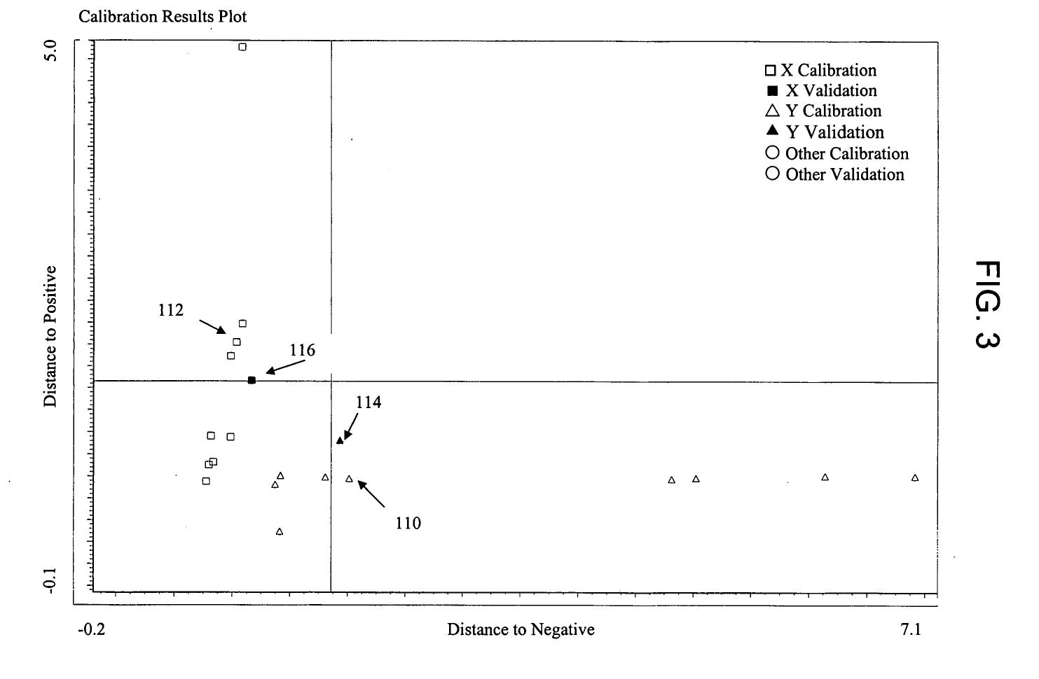

In a first example of the present invention, principal component analysis showed that the first nine (9) principal components accounted for over 99.5% of the total variance of the calibration tissue spectra. As illustrated in FIG. 3 for Example 1, a scatter plot of the calibration spectral data set after PCA transformation illustrates two distinct clusters of samples, a first cluster corresponding to BSE-positive samples (represented by triangular data points) and a second cluster corresponding to BSE-negative samples (represented by square data points). Accordingly, the calibration data set is used, after PCA transformation, to differentially identify BSE-positive and BSE-negative samples by comparing the unknown sample's spectral PC scores to the PC scores determined from the spectral data of known samples in the calibration set.

After all the calibration samples are scanned and the PC scores for each calibration sample in the calibration data set are calculated, the mean center PC scores are calculated for both the BSE-positive and BSE-negative cluster groupings, step 78. Alternatively, a mean centered spectra can be calculated for the entire calibration data set by averaging the absorbance/reflectivity values of the calibration samples at each wavelength to determine a mean spectra for each of the BSE-positive and BSE-negative cluster groupings, and the mean centered PC scores can be calculated therefrom. The mean center PC scores will be unique to each specific cluster grouping.

Further, the calibration set can be cross-validated, step 80, by calculating the Mahalanobis distances from each known sample in the calibration set to the mean center BSE-positive PC scores and the mean center BSE-negative PC scores of the remaining known samples in the calibration set (e.g. by rotating each cross-validated sample out of the calibration set and calculating the Mahalanobis distances for that sample from the mean center BSE-positive PC scores and the mean center BSE-negative PC scores of the remaining known samples in the calibration set.) As well known to those skilled in the art, the Mahalanobis distance is used for determining the “similarity” of a sample to a reference set, for example, by comparing the unknown sample's PC scores to the mean center PC scores of both the BSE-positive and the BSE-negative cluster groupings of the calibration data set.

As illustrated in FIG. 2B, to determine whether an unknown sample is BSE-positive or BSE-negative, the unknown sample (or, alternatively, a validation sample previously identified as BSE-positive or BSE-negative) is first scanned to obtain the sample's spectral information, steps 90 and 92. The PC scores of the unknown sample are then calculated. The Mahalanobis distances from both the mean center of the BSE-positive cluster grouping and the BSE-negative cluster groupings for the particular unknown sample are calculated from the calculated PC scores for the unknown sample, step 94. If the Mahalanobis distance from the unknown sample's PC scores to the mean center BSE-positive PC scores is closer than the Mahalanobis distance from the unknown sample's PC scores to the mean center BSE-negative PC scores, the unknown sample is classified as BSE-positive, step 96. Likewise, if the Mahalanobis distance from the unknown sample's PC scores to the mean center BSE-negative PC scores is closer than the Mahalanobis distance from the unknown sample's PC scores to the mean center BSE-positive PC scores, the unknown sample is classified as BSE-negative, step 96. (Reference is made to Examples 1 through 3 and FIGS. 3, 5 and 7 illustrating correctly classified validation samples).

If both Mahalanobis distances (to the mean center BSE-negative PC scores and to the mean center BSE-positive PC scores) calculated during cross-validation of the calibration samples, during a validation sample test or during an unknown sample test are greater than 3, the sample is considered a Mahalanobis outlier, step 96. Such Mahalanobis outliers are considered a <No Test> result. Mahalanobis outliers may be caused by improper sample trimming techniques or contamination/corruption of the tissue sample and may not result in a proper diagnosis. Thus, any samples tested via the method of the present invention that are Mahalanobis outliers can be removed from the calibration data set, and if the sample is a validation sample, will result in a <No Test> result.

The method of the present invention also includes updating the calibration data set with correctly diagnosed “unknown” bovine sample spectra in order to continually update the calibration set, step 100. This permits the calibration model to best account for possible variation, such as strain differentiation, typically found in naturally occurring BSE infections. As such, the calibration set for a given species can be continually optimized using samples that have been identified as BSE-positive and BSE-negative (and, if necessary, verified using another conventionally known BSE testing method). Thus, as described previously, calibration data sets can be developed and/or optimized not only for a particular bovine animal species, but also for a particular geographical area, region or state, or a specific chemical, physiological or physical characteristic of a BSE.

Accordingly, the diagnostic test/validated calibration model for BSE-positive and BSE-negative samples can be used at several locations or laboratories in order to provide remote testing capabilities for diagnosing a BSE infection. Once developed, the calibration model permits BSE testing of an unlimited number of samples without the need for purchasing chemicals, antibodies and/or other sample preparation materials. More particularly, the BSE blood test permits researchers, farmers, and/or ranchers the ability to correctly and rapidly classify an animal as BSE-positive or BSE-negative prior to extinguishing the animal. Thus, the method of the present invention provides a diagnostic test for ensuring a live animal and/or animal products, such as meat or milk, are disease free prior to entering the marketplace.

Sample Scoring.

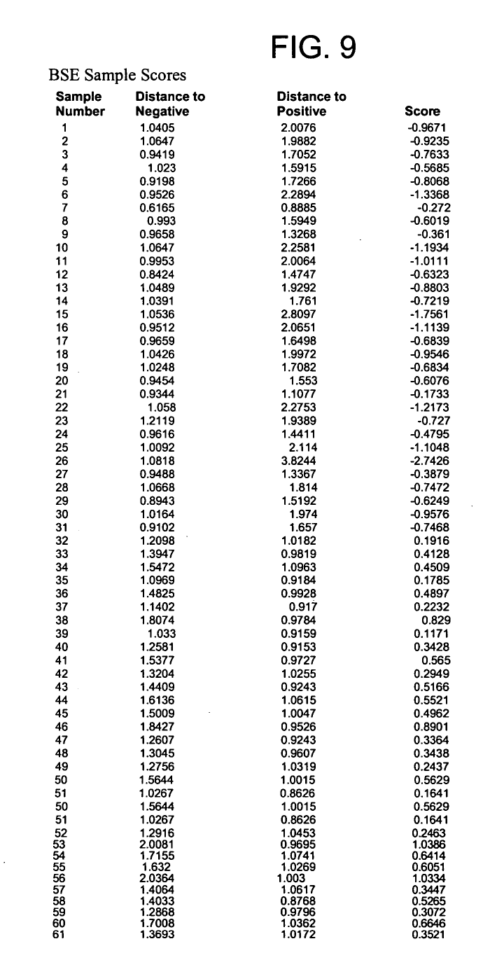

Samples tested in accordance with the present invention are scored with respect to a BSE-negative or BSE-positive result, step 98 of FIG. 2B. Sample scores are calculated from the Mahalanobis distances determined from the unknown sample PC scores to the mean center of the BSE-positive cluster grouping and the mean center BSE-negative cluster groupings. The score is calculated by subtracting the (Mahalanobis) distance to positive from the (Mahalanobis) distance to negative. (Reference is made to Example 5 and FIG. 9).

Any BSE-negative sample, as determined by the calibration model, having a calculated score ranging from between about −0.1000 to 0.0000 may be classified as <suspect positive>. For example a calculated score of −0.0900 would be classified as a <suspect positive> sample. Preferably, <suspect positive> samples are further tested by another BSE testing method such as by Western Blot or ELISA assay, step 102.

In general, sample scoring permits the diagnosis of the sample to be quantified, Thus, depending on the particular animal genotype or BSE strain, calculation of the sample score may be adjusted to increase the sensitivity of the test, perhaps, at the expense of specificity of the test. For example, sample scoring calculations may be adjusted to be more sensitive to, and therefore increase the incidence of, a <suspect positive> result, ensuring the number of false negative results of the test are substantially zero.

After testing is completed on a sample, or alternatively, a group of samples, diagnosis reports can be generated, step 104 of FIG. 2B. Such reports can contain animal information, the origin of the animal, animal owner, diagnosis, lot information, etc. in order to provide researchers or governments with information for tracking and monitoring the animal and/or animal byproducts.

Thus, with reference to the preceding, the present invention includes, in part, an infrared spectroscopic method for developing a reference or calibration model for use in differentially diagnosing naturally occurring bovine spongiform encephalopathy, comprising (1) obtaining a plurality of known BSE-positive and known BSE-negative untreated samples to form a calibration set; (2) analyzing each sample in the calibration set using an IR spectrometric method and obtaining calibration IR spectral data for each known sample in the calibration set; (3) applying a multivariate chemometric technique to the calibration spectral data for each sample to statistically differentiate the BSE-positive and BSE-negative calibration spectral data contained in the calibration set. The method can also include updating the calibration set with spectra of similar sample types that have been correctly diagnosed using the method of the present invention and verified via another BSE diagnostic technique. Certain embodiments of the present invention utilize whole blood, blood serum and/or blood plasma samples. In addition, tissue samples from the cortex or paracortex of a lymph node and/or obex tissue from the brain of the bovine animal can be used. Preferably, the sample is irradiated at wavenumbers from about 4500 cm⁻¹ to about 350 cm⁻¹.

In part, the present invention can include a method for diagnosing bovine spongiform encephalopathy comprising (1) providing a blood sample from a live bovine subject to determine whether the subject has a BSE infection, (2) obtaining IR spectral data from the sample, and (3) determining whether the sample is BSE-positive or BSE-negative by differentially comparing the spectral data of the sample with a calibration model comprising IR spectral data from a plurality of known BSE-positive and known BSE-negative samples.

In part, the present invention is a method for rapidly screening/classifying an unknown sample from a bovine subject as BSE-positive or BSE-negative. The method comprises (1) obtaining a sample from a bovine animal, (2) directing a beam of IR light at wavenumbers from about 7400 cm⁻¹ to about 350 cm⁻¹ to produce IR spectral data for the sample, (3) comparing the spectral data for the sample with a calibration set of infrared spectral data comprising both a BSE-positive and BSE-negative predictive model, and (4) determining whether variation in infrared absorption occurs in the sample, within at least one range of wavenumbers, due to the variation being characteristic of a positive indicator of BSE or a negative indicator of BSE.

The present invention also includes detecting a change in spectral response between diseased and normal samples to differentially diagnose BSE. The method includes (1) analyzing a substantially untreated, unknown sample selected from one of lymph tissue, obex tissue or blood using an IR spectroscopic method providing IR spectral data, (2) applying principal component analysis to the IR spectral data obtained from the sample and calculating the PCA scores for the unknown sample, and (3) differentially determining whether the unknown sample is positive or negative for BSE by comparing the PC scores for the sample with a calibration IR spectral data set comprising mean centered PC scores for known BSE-positive and BSE-negative samples in the calibration spectral data set. The method can further include confirming of a positive BSE diagnosis using a validated assay method such as Western Blot, ELISA or another immunohistochemistry assay.

Differentially determining whether the unknown sample is BSE-positive or BSE-negative can include calculation of the Mahalanobis distance from the unknown sample's PC scores to the mean centered PC scores for the BSE-positive and BSE-negative sample groupings in the calibration spectral data set and determining whether the Mahalanobis distance from the unknown sample's PC scores is closer to the mean center BSE-positive PC scores or closer to the mean center BSE-negative PC scores.

In part, the present invention includes a method of utilizing principal component analysis to differentially distinguish between BSE-positive and BSE-negative samples. The method consists essentially of obtaining calibration IR absorbance spectra for a plurality of untreated known BSE-positive and untreated known BSE negative samples, applying principal component analysis to the calibration IR absorbance spectra obtained for each of the known BSE-positive and known BSE-negative samples to statistically discriminate between the calibration IR absorbance spectra of the known BSE-positive samples and the known BSE-negative samples, calculating the BSE-positive mean center principal component scores for the known BSE-positive samples and calculating the BSE-negative mean center principal component scores for the known BSE-negative samples, obtaining IR absorbance spectral data from an untreated, unknown sample, performing principal component analysis on the unknown sample including determining the principal component scores of the unknown sample and calculating a Mahalanobis distance from the principal component scores of the unknown sample to the BSE-positive mean center principal component scores and calculating a Mahalanobis distance from the principal component scores of the unknown sample to the BSE-negative mean center principal component scores; whereby the unknown sample is classified as BSE-positive or BSE-negative.

In part, the present invention is directed to a method of providing a remote diagnostic test for BSE. The method includes providing a spectrometric instrument for analysis of bovine samples and equipping the spectrometric instrument with a validated BSE diagnostic test comprising a calibration model specific to at least one of the sample type, the geographic area, the gender or the breed of the bovine animal. The method can also include updating the diagnostic test by incorporating spectral data from correctly diagnosed samples into the calibration model.

The present invention also a method for fully automated BSE screening. The method includes (1) providing a plurality of samples, wherein each of said samples is contained within a receptacle and tagged with information such as a sample number, animal age, animal location and owner information, (2) automatically transferring an individual sample from the receptacle to the spectrometric instrument, (3) directing a beam of IR light at wavenumbers from about 7400 cm⁻¹ to about 350 cm⁻¹ to produce spectral data for the sample, (4) comparing the IR spectral data for the sample with a calibration model for BSE, (5) recording test result/diagnosis information for the sample and (6) cleaning the spectrometric instrument prior to analysis of a subsequent sample.

It will be apparent to those skilled in the art that the methods of the present invention may be capable of diagnosing new variant Creutzfeld-Jakob (vCJD) disease in humans. In particular, insomuch as vCJD is caused by ingesting meat from BSE infected cows, molecular alterations in a human, similar to those present in a bovine animal, occurring before the onset of clinical vCJD disease may be present in the blood of humans. Accordingly, the present invention can include development of a calibration model for vCJD and method of diagnosing a patient with vCJD utilizing a blood sample therefrom.

Examples of the Invention.

The following non-limiting examples and data illustrate various aspects and features of the present invention, including the surprising and unexpected results obtained thereby. It should, of course, be understood that these examples are included for illustrative purpose only and that the invention is not limited to the particular tissue or biological fluid samples, collection conditions, species or the like set forth herein. Comparable utility and advantages can be realized using various other data analysis methodologies and/or embodiments consistent with the scope of this invention.

Test Equipment

All samples in Examples 1-3 were scanned using a lab grade FTIR spectrometer (Nexus 670 by Thermo Nicolet, Inc., Madison, Wis.) including a standard mid-infrared source (7400-350 cm⁻¹, res. 0.125 cm⁻¹) and a DLaTGS detector (deuterated trigycine sulphate doped with L-alanine) with KBr window and was equipped with a DuraSampLIR II™ 3-Reflection Diamond/ZnSe (SensIR Technologies, Danbury, Conn.). However, any commercially available infrared spectrophotometers may be utilized. Data collection and manipulation software OMNIC (® 2002 Thermo Electron Corporation) including TQ Analyst software (® 2002 Thermo Electron Corporation), capable of performing principal component analysis, was included with the instrument and used to perform the statistical analysis. However, any computer program known to those skilled in the art may be used to carry out the statistical methods taught by the present invention.

### EXAMPLE 1

### Development of a Calibration Model for Diagnosing Bovine Spongiform Encephalopathy in Bovine Blood Serum

Sample Sources

With respect to the calibration model data, nine known BSE positive and nine known BSE negative blood serum samples were obtained from the Veterinary Laboratories Agency, an executive agency of the Department of Environment, Food and Rural Affairs, in the United Kingdom. Validation samples consisted of one known BSE positive and one known BSE negative blood serum samples also obtained from the Veterinary Laboratories Agency. In all samples, a BSE diagnosis was predetermined using standard gold standard methods such as by Western Blot or ELISA assays.

Blood serum samples were previously frozen at −70° C. and allowed to thaw prior to testing. It will be appreciated by those skilled in the art that although previously frozen samples are used in the present example, freshly drawn blood samples from live bovine animals or freshly drawn samples from animals prior to slaughtering/sacrificing can be used in the present invention.

Testing

Approximately about 10 μl of untreated, blood serum from each sample subject was utilized. Samples were pipetted directly onto the optical platform of the instrument. Each sample in both the calibration set and the validation set were scanned 64 times and co-added to increase the signal to noise ratio, thereby enhancing the spectral absorbance features of the sample. Complete scanning of each sample (including the 64 scans of the same sample) was performed in less than about one minute. The FTIR data collection software, OMNIC/TQ Analyst (® 2002 Thermo Electron Corporation), collected the dispersive spectra for each sample.

FTIR Calibradon for BSE

Second derivatives were calculated from the original spectra for each sample in the calibration data set using a Savitzky-Golay smoothing algorithm using the aforementioned software. Calibration of the test for BSE was then performed using discriminate principal component analysis performed with the analysis software described herein. It will be appreciated by those skilled in the art that other multivariate chemometric techniques may be used for data analysis in developing the calibration/reference data set, including but not limited to, Partial Least Squares Regression (PLS), Principal Component Regression (PCR), Multilinear Regression Analysis (MLR) and Discriminant Analysis.

The software was calibrated with the known BSE-positive and BSE-negative samples to develop a calibration model specific for BSE infections in blood serum taken from bovine animals. Principal component analysis was performed on the calibration spectral data over the range from about 4500 cm⁻¹ to about 350 cm⁻¹ for each blood serum sample in the calibration data set. As illustrated in Table 1, nine (9) principal components, accounting for over 99.5% of the variance in the calibration spectral data set were used for the calibration model.

TABLE 1
PRINCIPAL COMPONENT ANALYSIS
OF CALIBRATION DATA SET

| PRINCIPAL | | |
|---|---|---|
| COMPONENT | % VARIANCE | CUMULATIVE VARIANCE |
| 1 | 94.687 | 94.687 |
| 2 | 2.821 | 97.507 |
| 3 | 0.884 | 98.392 |
| 4 | 0.499 | 98.891 |
| 5 | 0.222 | 99.113 |
| 6 | 0.171 | 99.284 |
| 7 | 0.152 | 99.436 |
| 8 | 0.132 | 99.568 |
| 9 | 0.098 | 99.667 |

A set of PC scores for each sample in the calibration spectral data set was calculated. In addition, the mean center PC scores were generated for the samples known to be positive for BSE and the samples known to be negative for BSE.

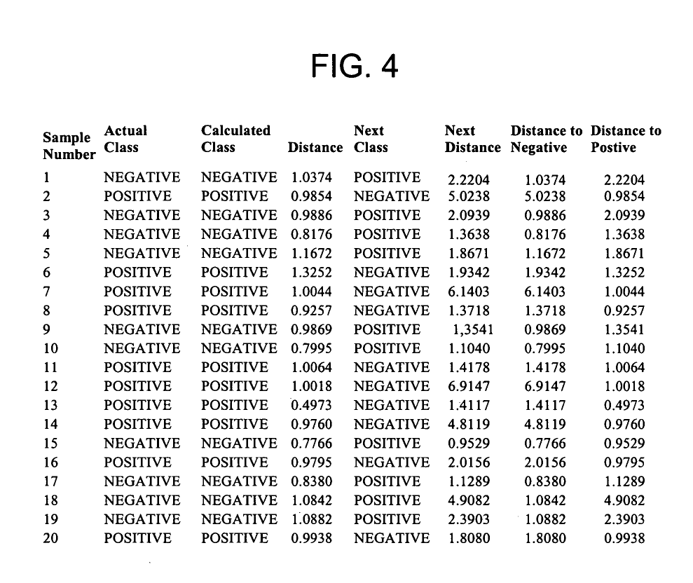

The calibration set was cross-validated by calculating the Mahalanobis distances from each known sample in the calibration set to the mean center BSE-positive PC scores and the mean center BSE-negative PC scores of the remaining known samples in the calibration set (e.g. by rotating each cross-validated sample out of the calibration set and calculating the Mahalanobis distances for that sample from the mean center BSE-positive PC scores and the mean center BSE-negative PC scores of the remaining known samples in the calibration set.) As illustrated in FIG. 3, a scatter plot of the Mahalanobis distances for each calibration sample spectra depicts two distinct clusters of calibration data points (unshaded points), a first cluster grouping 110 corresponding to BSE-positive samples (triangular unshaded points) and a second cluster grouping 112 corresponding to BSE-negative samples (square unshaded points). FIG. 4 contains a tabular representation of the calibration data samples (sample numbers 1 through 4 and 7 through 20) illustrating the actual class and the calculated class for each sample of the calibration data set. All calibration samples were correctly cross-validated as either BSE-positive or BSE-negative.

Consistent with the broader aspects of the invention, correlation between the present testing methodology and a validated TSE test will be evaluated by the kappa calculation; and agreement of less than 96% will result in recalibration of the system using additional refinement of the calibration spectrum examined, in particular, the calibration will be adjusted so that the sensitivity of the test approaches 100%. A slight decrease in test specificity (resulting in a small increase in false positive samples) is preferred in a pre-clinical screening test for TSEs. All TSE-positive diagnosis can be confirmed by IHC or other validated diagnostic test.

Validation of the Calibration Set for the Method for Detecting BSE in Blood Serum

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined from the PC scores of validation sample and the mean center PC scores of the BSE-positive cluster grouping of the calibration data set. Likewise, the Mahalanobis distance for each validation sample in the validation set was determined from the PC scores of validation sample and the mean center PC scores of the BSE-negative cluster grouping of the calibration data set. If the Mahalanobis distance from the validation sample's PC scores to the mean center BSE-positive PC scores was closer than the Mahalanobis distance from the validation sample's PC scores to the mean center BSE-negative PC scores, the validation sample was classified as positive for BSE. Likewise, if the Mahalanobis distance from the validation sample's PC scores to the mean center BSE-negative PC scores was closer than the Mahalanobis distance from the validation sample's PC scores to the mean center BSE-positive PC scores, the validation sample was classified as BSE-negative. FIG. 3 includes the Mahalanobis distances for each validation sample (shaded points), along with the calibration data points (unshaded points). As can be seen, each validation sample falls into one of the two distinct clusters of groups, corresponding to either a BSE-positive disease state (sample number 6, triangular shaded point 114) or BSE-negative disease state (sample number 5, square shaded point 116).

As illustrated in FIG. 4, each validation sample was correctly diagnosed, thus, use of the present methodology revealed 100% agreement of the validation test results with the gold standard results for each sample in the validation set.

### EXAMPLE 2

### Development of a Calibration Model for Diagnosing Bovine Spongiform Encephalopathy in Bovine Brain Samples

Tissue Handling

Brain samples containing obex tissue and/or spinal tissue is obtained from postmortem bovine animals, as illustrated in FIGS. 1B and 1C.

An approximately about 1 cm×about 1 cm×about 0.2 cm section of tissue from each subject was collected. Samples were trimmed, if required, to maximize obex/spinal cord tissue within the sample. The exposed tissue was placed directly on the optical bench of the FTIR instrument without further work-up.

Sample Sources

With respect to the calibration model samples, 13 known BSE positive and 19 known BSE negative samples were obtained from the Veterinary Laboratories Agency, an executive agency of the Department of Environment, Food and Rural Affairs, in the United Kingdom. Validation samples consisted of one known BSE positive and one known BSE negative sample also obtained from the Veterinary Laboratories Agency. In all samples, a BSE diagnosis was predetermined using standard gold standard methods such as by Western Blot or ELISA assays.

Testing

Each sample in both the calibration set and the validation set were scanned 64 times and co-added to increase the signal to noise ratio, thereby enhancing the spectral absorbance features of the sample. Complete scanning of each sample (including the 64 scans of the same sample) was performed in less than about one minute. The FTIR data collection software, OMNIC/TQ Analyst (® 2002 Thermo Electron Corporation), collected the dispersive spectra for each sample.

FTIR Calibration for BSE

As in Example 1, second derivatives were calculated from the original spectra for each sample in the calibration data set using a Savitzky-Golay smoothing algorithm using the aforementioned software. Calibration of the test for BSE was then performed using discriminate principal component analysis performed with the analysis software described herein.

The software was calibrated with the known BSE-positive and BSE-negative samples to develop a calibration model specific for BSE infections in brain tissue taken from bovine animals. Principal component analysis was performed on the calibration spectral data over the range from about 4500 cm⁻¹ to about 350 cm⁻¹ for each sample in the calibration data set. As illustrated in Table 2, fifteen (15) principal components, accounting for over 99.9% of the variance in the calibration spectral data set were used for the calibration model.

TABLE 2
PRINCIPAL COMPONENT ANALYSIS
OF CALIBRATION DATA SET

| PRINCIPAL | | |
|---|---|---|
| COMPONENT | % VARIANCE | CUMULATIVE VARIANCE |
| 1 | 95.458 | 95.458 |
| 2 | 3.276 | 98.734 |
| 3 | 0.673 | 99.407 |
| 4 | 0.359 | 99.766 |
| 5 | 0.133 | 99.898 |
| 6 | 0.050 | 99.949 |
| 7 | 0.019 | 99.967 |
| 8 | 0.011 | 99.978 |
| 9 | 0.006 | 99.984 |
| 10 | 0.003 | 99.987 |
| 11 | 0.003 | 99.990 |
| 12 | 0.002 | 99.992 |
| 13 | 0.001 | 99.993 |
| 14 | 0.001 | 99.994 |
| 15 | 0.001 | 99.995 |

A set of PC scores for each tissue sample in the calibration spectral data set was calculated. In addition, the mean center PC scores were generated for the samples known to be positive for BSE and the samples known to be negative for BSE.

The calibration set was cross-validated by calculating the Mahalanobis distances from each known sample in the calibration set to the mean center BSE-positive PC scores and the mean center BSE-negative PC scores of the remaining known samples in the calibration set (e.g. by rotating each cross-validated sample out of the calibration set and calculating the Mahalanobis distances for that sample from the mean center BSE-positive PC scores and the mean center BSE-negative PC scores of the remaining known samples in the calibration set.)

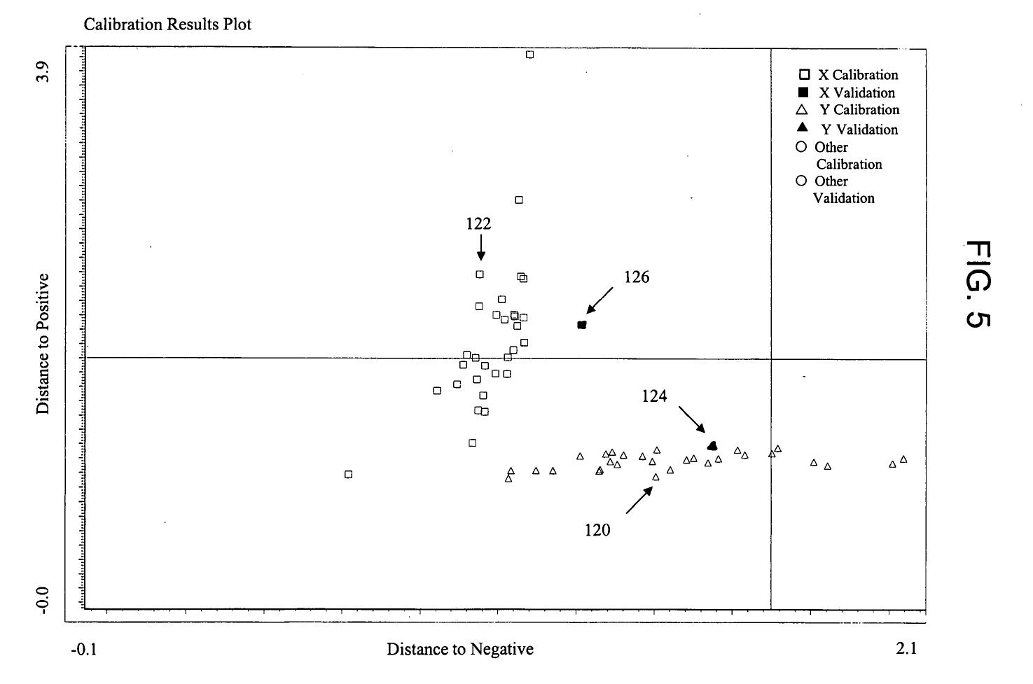

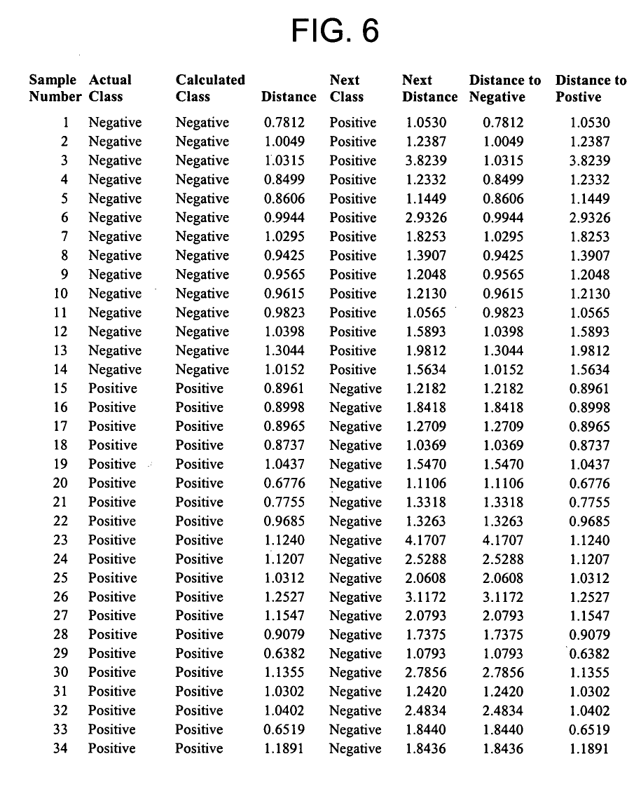

As illustrated in FIG. 5, a scatter plot of the Mahalanobis distances for each calibration sample spectra depicts two distinct clusters of calibration data points (unshaded points), a first cluster grouping 120 corresponding to BSE-positive tissue samples (triangular unshaded points) and a second cluster grouping 122 corresponding to BSE-negative tissue samples (square unshaded points). FIG. 6 contains a tabular representation of the calibration data samples (sample numbers 1 through 12, 14 through 23, and 25 through 34) illustrating the actual class and the calculated class for each sample of the calibration data set. All calibration samples were correctly cross-validated as either BSE-positive or BSE-negative.

Validation of the Calibration Set for the Method for Detecting BSE in Brain Samples

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined from the PC scores of validation sample and the mean center PC scores of the BSE-positive cluster grouping of the calibration data set. Likewise, the Mahalanobis distance for each validation sample in the validation set was determined from the PC scores of validation sample and the mean center PC scores of the BSE-negative cluster grouping of the calibration data set. Each validation sample was classified as either positive or negative for BSE, as described in Example 1. FIG. 5 includes the Mahalanobis distances for each validation sample (shaded points), along with the calibration data points (unshaded points). As can be seen, each validation sample falls into one of the two distinct clusters of groups, corresponding to either a BSE-positive disease state (sample number 24, triangular shaded point 124) or a BSE-negative (sample number 13, square shaded point 126).

As illustrated in FIG. 6, each validation sample was correctly diagnosed, thus, use of the present methodology revealed 100% agreement of the validation test results with the gold standard results for each sample in the validation set.

### EXAMPLE 3

### Development of a Calibration Model for Diagnosing Bovine Spongiform Encephalopathy in Bovine Lymph Tissue

Tissue Handling

A mesenteric lymph node from each subject was sectioned to expose the cortex and paracortex of the node, as illustrated in FIG. 1A.

An approximately about 1 cm×about 1 cm×about 0.2 cm section of a lymph node from a subject was then collected from the cortex or paracortex of the lymph node to obtain sample sections containing primary and/or secondary follicles. Samples were trimmed, if required, to minimize medullary tissue within the sample. The exposed tissue was placed directly on the optical bench of the FTIR instrument without further work-up.

Sample Sources

With respect to the calibration model samples, 29 known BSE positive and 30 known BSE negative samples were obtained from the Veterinary Laboratories Agency, an executive agency of the Department of Environment, Food and Rural Affairs, in the United Kingdom. Validation samples consisted of one known BSE positive and one known BSE negative sample also obtained from the Veterinary Laboratories Agency. In all samples, a BSE diagnosis was predetermined using standard gold standard methods such as by Western Blot or ELISA assays.

Testing

Each sample in both the calibration set and the validation set were scanned and original spectra was collected as described with reference to Examples 1 and 2.

FTIR Calibration for BSE

As in Example 1, second derivatives were calculated from the original spectra for each sample in the calibration data set using a Savitzky-Golay smoothing algorithm using the aforementioned software. Calibration of the test for BSE was then performed using discriminate principal component analysis performed with the analysis software described herein.

The software was calibrated with the known BSE-positive and BSE-negative samples to develop a calibration model specific for BSE infections in brain tissue taken from bovine animals. Principal component analysis was performed on the calibration spectral data over the range from about 4500 cm⁻¹ to about 350 cm⁻¹ for each sample in the calibration data set. As illustrated in Table 3, ten (10) principal components, accounting for over 99.9% of the variance in the calibration spectral data set were used for the calibration model.

TABLE 3
PRINCIPAL COMPONENT ANALYSIS
OF CALIBRATION DATA SET

| PRINCIPAL | | |
|---|---|---|
| COMPONENT | % VARIANCE | CUMULATIVE VARIANCE |
| 1 | 94.564 | 95.458 |
| 2 | 2.972 | 97.536 |
| 3 | 1.558 | 99.094 |
| 4 | 0.518 | 99.611 |
| 5 | 0.217 | 99.829 |
| 6 | 0.049 | 99.877 |
| 7 | 0.031 | 99.908 |
| 8 | 0.019 | 99.928 |
| 9 | 0.018 | 99.946 |
| 10 | 0.015 | 99.961 |

A set of PC scores for each tissue sample in the calibration spectral data set was calculated. In addition, the mean center PC scores were generated for the samples known to be positive for BSE and the samples known to be negative for BSE.

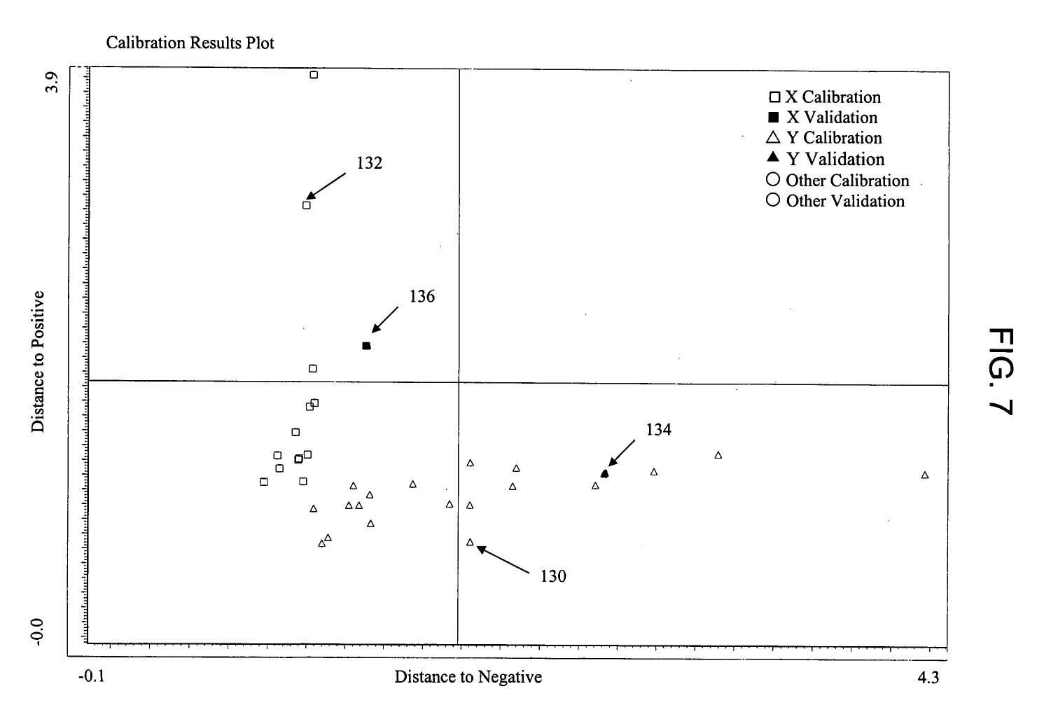

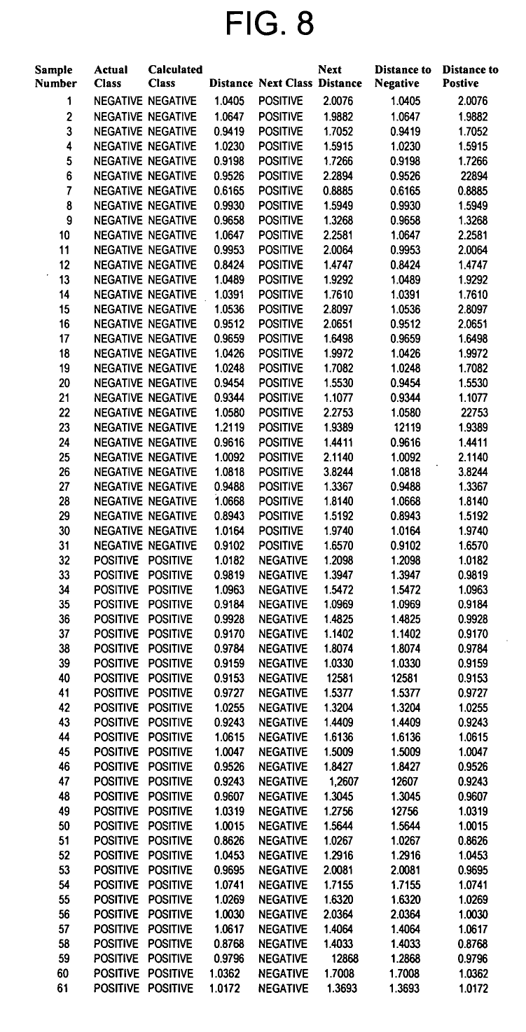

The calibration set was cross-validated by calculating the Mahalanobis distances from each known sample in the calibration set to the mean center BSE-positive PC scores and the mean center BSE-negative PC scores of the remaining known samples in the calibration set (e.g. by rotating each cross-validated sample out of the calibration set and calculating the Mahalanobis distances for that sample from the mean center BSE-positive PC scores and the mean center BSE-negative PC scores of the remaining known samples in the calibration set.) As illustrated in FIG. 7, a scatter plot of the Mahalanobis distances for each calibration sample spectra depicts two distinct clusters of calibration data points (unshaded points), a first cluster grouping 130 corresponding to BSE-positive tissue samples (triangular unshaded points) and a second cluster grouping 132 corresponding to BSE-negative tissue samples (square unshaded points). FIG. 8 contains a tabular representation of the calibration data samples (sample numbers 1 through 22, 24 through 33, and 35 through 61) illustrating the actual class and the calculated class for each sample of the calibration data set. All calibration samples were correctly cross-validated as either BSE-positive or BSE-negative.

Validation of the Calibration Set for the Method for Detecting BSE in Lymph Tissue

Analysis of Validation Spectral Data Using the Predictive Model

Second derivatives were calculated from the original spectra for each sample in the validation data set using a Savitzky-Golay smoothing algorithm using the aforementioned software. Principal component analysis was then performed on the spectral data for each validation sample using the ten principal components identified in the calibration model. These spectral features were analyzed by PCA and the PC scores for each validation sample were determined.

The Mahalanobis distance for each validation sample in the validation set was determined from the PC scores of validation sample and the mean center PC scores of the BSE-positive cluster grouping of the calibration data set. Likewise, the Mahalanobis distance for each validation sample in the validation set was determined from the PC scores of validation sample and the mean center PC scores of the BSE-negative cluster grouping of the calibration data set. Each validation sample was classified as either positive or negative for BSE, as described in Example 1. FIG. 7 includes the Mahalanobis distances for each validation sample (shaded points), along with the calibration data points (unshaded points). As can be seen, each validation sample falls into one of the two distinct clusters of groups, corresponding to either a BSE-positive disease state (sample number 34, triangular shaded point 134) or a BSE-negative disease state (sample number 23, square shaded point 136).

As illustrated in FIG. 7, each validation sample was correctly diagnosed, thus, use of the present methodology revealed 100% agreement of the validation test results with the gold standard results for each sample in the validation set.

### EXAMPLE 5

### Sample Scoring

Each calibration and validation sample in Example 4 was scored for the presence of BSE in the sample. As illustrated in FIG. 9, a score was calculated for each sample by subtracting the distance to positive from the distance to negative. If a sample score falls within the range of −0.1000 to 0.0000, the sample is classified as <suspect positive>. As shown in FIG. 9, none of the samples tested were classified as <suspect positive> samples.

1. A method for detecting bovine spongiform encephalopathy (BSE) in a live bovine subject, said method comprising:
providing a blood sample from the bovine subject;
obtaining at least one set of IR spectral data from the blood sample; and
comparing the IR spectral data of the sample with a calibration model comprising calibration IR spectral data from a plurality of known BSE-positive and known BSE-negative blood samples and classifying the sample as BSE-positive or BSE-negative.

2. The method of claim 1, wherein the blood sample is selected from whole blood, blood plasma and blood serum.

3. The method of claim 1, wherein the blood sample is substantially untreated prior to obtaining the spectral data.

4. The method of claim 1, wherein the spectral data of the blood sample and the calibration spectral data is obtained at wavenumbers from about 4500 cm⁻¹ to about 350 cm⁻¹.

5. The method of claim 1, wherein the spectral data of the blood sample and the calibration spectral data is obtained using an absorption or reflectance spectrometric method.

6. The method of claim 1, wherein the calibration model comprises a BSE-positive predictive model and a BSE-negative predictive model developed by means of multivariate analysis utilizing the calibration spectral data from each of the plurality of known BSE-positive and known BSE-negative blood samples.

7. A method for developing a calibration model for use in differentially diagnosing naturally occurring bovine spongiform encephalopathy, said method comprising:
obtaining a plurality of known BSE-positive and known BSE-negative untreated samples to form a calibration set;
analyzing each of the known BSE-positive samples and each of the known BSE-negative samples in the calibration set using an IR spectrometric method;
obtaining at least one set of calibration IR spectral data for each of the known BSE-positive and known BSE-negative samples in the calibration set; and
applying a multivariate chemometric technique to the calibration IR spectral data for each of the known BSE-positive samples and each of the known BSE-negative samples to statistically differentiate the BSE-positive and BSE-negative calibration IR spectral data contained in the calibration set.

8. The method of claim 7, wherein each of the known BSE-positive and the known BSE-negative samples are selected from the group consisting of brain tissue, lymph tissue and blood.

9. The method of claim 7, wherein the spectrometric method is an absorption or reflectance spectrometric method.

10. The method of claim 7, wherein each of the known BSE-positive and known BSE-negative samples in the calibration set is analyzed at wavenumbers from about 4500 cm⁻¹ to about 350 cm⁻¹.

11. The method of claim 7, wherein principal component analysis is applied to the calibration spectral data obtained for each of the known BSE-positive samples and each of the known BSE-negative samples to statistically differentiate the BSE-positive and BSE-negative calibration spectral data contained in the calibration set.

12. The method of claim 11, further comprising calculating BSE-positive mean center principal component scores for the known BSE-positive tissue samples and BSE-negative mean center principal component scores for the known BSE-negative samples.

13. The method of claim 12, further comprising analyzing an unknown sample of the same type as each of the known BSE-positive samples and each of the known BSE-negative samples using a spectrometric method, obtaining at least one set of spectral data for the unknown sample, calculating the principal component scores of the unknown sample, comparing the principal component scores of the unknown sample to the BSE-positive mean center principal component scores and the BSE-negative mean center principal component scores; and classifying the unknown tissue sample as one of BSE-positive and BSE-negative.

14. The method of claim 13, further comprising calculating a Mahalanobis distance from the principal component scores of the unknown sample to each of the BSE-positive and BSE-negative mean centered principal component scores, wherein the unknown tissue sample is classified as one of BSE-positive, BSE-negative and a Mahalanobis outlier.

15. The method of claim 7, further comprising analyzing an unknown, untreated sample of the same type as each of the known BSE-positive samples and each of the known BSE-negative samples using a spectrometric method, obtaining at least one set of spectral data for the unknown sample, applying a multivariate chemometric technique to the spectral data for the unknown sample, comparing the spectral data of the unknown sample to the calibration spectral data of the known BSE-positive and the known BSE-negative tissue samples, whereby said unknown sample is classified as one of BSE-positive and BSE-negative.

16. The method of claim 13, further comprising updating the calibration set with the spectral data obtained for the unknown sample.

17. A method of diagnosing bovine spongiform encephalopathy (BSE) in a bovine animal, said method comprising:
obtaining at least one set of IR spectral data from an untreated, unknown sample; and
comparing IR spectral data of the sample with a calibration model comprising calibration IR spectral data from a plurality of known BSE-positive and known BSE-negative samples of similar type as the untreated, unknown sample to determine whether the sample is BSE-positive or BSE-negative.

18. The method of claim 17, wherein the untreated, unknown sample is selected from the group consisting of lymph tissue, brain tissue and blood.

19. The method of claim 17, wherein infrared absorbance spectra for the unknown sample and each of the known samples are obtained at wavenumbers from about 4500 cm⁻¹ to about 350 cm⁻¹.

20. The method of claim 17, further comprising subjecting the calibration spectral data of the calibration model to a spectral data compression technique to identify changes in the spectral data corresponding to one of a BSE-positive disease state and a BSE-negative disease state.

21. The method of claim 20, wherein the spectral data compression technique selected from the group consisting of Principal Component Analysis (PCA), Partial Least Squares Regression (PLS), Principal Component Regression (PCR), Multiple Linear Regression (MLR) and Discriminant Analysis.

22. The method of claim 20, further comprising subjecting the spectral data of the untreated, unknown sample to a spectral data compression technique, wherein the compressed spectral data of the untreated, unknown sample is compared to the calibration model, wherein the untreated, unknown sample is classified as BSE-positive or BSE-negative.

23. A method of classifying an unknown sample from a bovine animal as BSE-positive or BSE-negative, said method comprising:
directing a beam of IR light at wavenumbers from about 7400 cm⁻¹ to about 350 cm⁻¹ at a sample to produce IR spectral data for the sample, wherein the sample is selected from a brain sample, a lymph sample and a blood sample;
subjecting the IR spectral data of the sample to multivariate analysis;
comparing the analyzed IR spectral data of the sample to each of a BSE-positive and a BSE-negative predictive model, each of the predictive models comprising calibration IR spectral data; and
classifying the sample as one of a BSE-positive or a BSE-negative disease condition.

24. The method of claim 23, wherein classifying the sample comprises comparing the spectral data of the sample to the calibration spectral data and determining whether the variation in absorption observed in the spectral data of the sample indicates one of a BSE-positive or a BSE-negative condition.

25. The method of claim 23, wherein each of the BSE-positive and the BSE-negative predictive models are obtained through multivariate analysis of the calibration spectral data comprising known BSE-positive and known BSE-negative samples to distinguish changes in the calibration spectral data corresponding to one of a BSE-positive and a BSE negative disease state.

26. A method for screening unknown samples for bovine spongiform encephalopathy, said method comprising:
analyzing an untreated sample selected from lymph tissue, brain tissue or blood using an IR spectroscopic method providing IR spectral data;
applying principal component analysis to the IR spectral data obtained from the unknown sample and calculating the principal component scores for the unknown sample; and
differentially determining whether the sample is BSE-positive or BSE-negative by comparing the principal component scores for the unknown sample with a calibration IR spectral data set comprising a set of mean centered principal component scores for each of a known BSE-positive sample grouping and a known BSE-negative sample grouping.

27. The method of claim 26, wherein spectral data for the unknown sample and each of the known samples are obtained at wavenumbers from about 4500 cm⁻¹ to about 500 cm⁻¹.

28. The method of claim 26, wherein differentially determining whether the unknown sample is BSE-positive or BSE-negative includes calculating a positive Mahalanobis distance from the principal component scores of the unknown sample to the mean centered principal component scores for the BSE-positive sample grouping and calculating a negative Mahalanobis distance from the principal component scores of the unknown sample to the mean centered principal component scores for the BSE-negative sample grouping; and
determining whether one of the positive and the negative Mahalanobis distances of the unknown sample is closer to the mean center BSE-positive principal component scores or closer to the mean center BSE-negative principal component scores.

29. The method of claim 28, further comprising calculating a BSE diagnostic score for the unknown sample by subtracting the negative Mahalanobis distance from the positive Mahalanobis distance.

30. The method of claim 26, further comprising confirming of a BSE-positive diagnosis using a secondary diagnosis method selected from the group consisting of immunohistochemistry assay, Western Blot assay, microscopic examination and ELISA assay.

31. A method of using principal component analysis to differentiate between BSE-positive and BSE-negative samples, said method comprising:
providing a calibration set comprising a plurality of known, untreated BSE-positive and known, untreated BSE-negative samples;
obtaining at least one set of calibration IR spectral data for each of the known BSE-positive samples and each of the known BSE-negative samples in the calibration set;
calculating a set of principal component scores for each of the known BSE-positive samples and each of the known BSE-negative samples in the calibration data set;
calculating a set of BSE-positive mean center principal component scores for the known BSE-positive samples and calculating a set of BSE-negative mean center principal component scores for the known BSE-negative samples;
obtaining at least one set of IR spectral data for an unknown sample;
calculating a set of principal component scores from the IR spectral data of the unknown sample;
determining a first distance from the set principal component scores of the unknown sample to the set of BSE-positive mean center principal component scores and determining a second distance from the set of principal component scores of the unknown sample to the set of BSE-negative mean center principal component scores, whereby the disease state of the unknown sample is determined.

32. The method of claim 31, wherein each of the known samples and the unknown sample are raw and untreated samples selected from blood, lymph tissue and brain tissue.

33. The method of claim 31, wherein the set of principal component scores for each of the known BSE-positive samples and each of the known BSE-negative samples in the calibration data set correspond to substantially over about 97% of the variance within the calibration IR spectral data.

34. The method of claim 31, further comprising calculating a diagnosis score of the unknown sample comprising subtracting the first distance from the second distance, and classifying the unknown sample as <suspect positive> if the diagnosis score falls within the range between about −0.1000 to about 0.0000,
